(19) United States
(12) Patent Application Publication
(10) Pub. No.: US 2023/0194527 A1
(43) Pub. Date: Jun. 22, 2023

(54) A Method For Virus and Biomarker Detection

(71) Applicant: National University of Singapore, Singapore (SG)

(72) Inventors: Jie Yan, Singapore (SG); Rong Li, Singapore (SG); Chen Lu, Singapore (SG); Xiaodan Zhao, Singapore (SG); Yu Zhou, Singapore (SG); Shimin Le, Singapore (SG)

(21) Appl. No.: 17/924,958

(22) PCT Filed: May 12, 2021
(86) PCT No.: PCT/SG2021/050264

§ 371 Date: Nov. 11, 2022

(30) Foreign Application Priority Data
May 12, 2020 (SG) 10202004395T

(51) Int. Cl. G01N 33/569; G01N 33/543

(52) U.S. Cl. CPC G01N 33/56983; G01N 33/54326

(57) ABSTRACT

Disclosed herein is a method of detecting the presence or absence of an analyte in a test sample, based on the use of motion resistant particle for example non-magnetic particle coated with first sensing element for the analyte and force driven particle for example superparamagnetic particle coated with a second sensing element for the analyte so as to form a motion resistant particle-analyte-force driven particle conjugate. A viscous medium is added to the mixture and a force such as magnetic force is applied to provide separation of the conjugate for quantification. Disclosed herein are also a sensing kit, a system for detecting the presence or absence of an analyte in a test sample and the use of the kit or the system thereof.

### REFERENCES TO RELATED APPLICATION

This application claims priority to Singapore application number 10202004395T filed on 12 May 2020, the disclosure of which is hereby incorporated by reference.

### FIELD OF THE INVENTION

The present invention relates generally to the field of virus and biomarker detection. In particular, the present invention relates to the use of nanometer-to-micrometer particles for analyte detection under a force (such as a mechanical force, which in turn, can be exerted by means such as, but not limited to, magnetic field).

### BACKGROUND ART

For microorganism and virus detection, current viral RNA or DNA based technologies are available such as RT-PCR based assays (Ian M. Mackay et al., Nucleic Acids Res, 2002, 30 (6): 1292-1305). Current antibody-based detections are available, such as 1) enzyme-linked immunosorbent assay (ELISA, Eva Engvall and Peter Perlmann, J Immunol, 1972, 109 (1): 129-135), 2) immunogold labelling based assays, and 3) chemiluminescent microparticle immunoassay (Abbott Laboratories Inc.). Other diagnostic methods such as surface plasmon resonance (SPR) based assays depend on surface plasmonic resonance signal. The interference from surface non-specific interaction in the SPR based assays may result in false-positive signals. These methods are often expensive, time costly, and mainly are central lab-based assays. In addition to the above-mentioned technologies, lateral flow-based assays provide a convenient, rapid and low-cost diagnostic method. However, such methods often suffer from insufficient sensitivity or specificity.

There is a need to develop a detection method for viruses and biomarkers with a different detection principle that overcome or ameliorate one or more of the limitations with the existing technologies.

### SUMMARY

Here we describe a universal, fluorescence label-free method for detecting viruses in a wide range of environmental conditions, with a single-particle sensitivity and enhanced specificity based on mechanical selection of specific interactions. The novel method enables fast, low-cost point-of-care diagnosis and home-based self-diagnosis of specific viruses and viral infection induced antibodies in suspected human samples. It also serves as a novel platform for discovering viral entry inhibitors.

The principle of the method described here also applies for fast, accurate and sensitive detection of any other multivalent targets such as biomarkers, exosomes, antibodies, pathogen-associated molecules, pollutants, etc. The method serves as a novel platform for disease diagnostics, biologics drug discovery, and environment monitoring.

In a first aspect, the present disclosure relates to a method of detecting the presence or absence of an analyte in a test sample comprising the steps of:
a) incubating a plurality of motion-resistant particles and a plurality of force-driven particles with said test sample to form a test mixture, wherein said motion-resistant particles are coated with a first sensing element and said force-driven particles are coated with a second sensing element, both first and second sensing elements are capable of specific binding with the analyte, wherein when the analyte is present in the test sample, said analyte binds with said first and second sensing elements on said motion-resistant particles and said force-driven particles, respectively, to form bound motion-resistant /analyte/ force-driven particle conjugates;
b) adding the test mixture of step a) to a viscous medium and applying a force to the force-driven particles to separate said bound motion-resistant/analyte/force-driven particle conjugates, when present, from the unbound motion resistant particles and unbound force-driven particles.

Advantageously, the method is universal and does not require the use of fluorescence labels for detecting a range of analytes including viruses in a wide range of environmental conditions. Mechanical selection achieved by the force application (such as mechanical force) further enhances the specificity of detection, as the specifically linked particle-particle conjugates dissociate under the application of the mechanical force slower than the non-specific ones. Counting the amount of specifically linked particle-particle conjugates results in the detection, with single-particle sensitivity and accuracy of detection. The method enables fast diagnosis of viral infections and may serve as a platform for discovering viral entry inhibitors. This is highly advantageous for clinical settings which require fast diagnosis to be carried out. Further, due to the ease of using the method, the method can be used as a home-based self-diagnosis of viral infection.

Further advantageously, when applied to virus detection, the method does not require nucleic acids extraction from virus, reverse transcription (RT) for RNA viruses or PCR amplification, therefore avoiding significant loss of sample size during multiple-step procedures. The method can also be used for enriching the analyte in a low-concentration sample for downstream analysis such as sequencing. The method can be carried out on a small sample size, such as a finger-prick blood droplet. Due to its mechanically enhanced specificity and single-particle sensitivity, it can detect analyte in low concentrations, such as samples having a concentration in the picomolar to femtomolar.

In a second aspect, the present disclosure relates to a sensing kit comprising
a plurality of motion-resistant particles; and
a plurality of force-driven particles,
wherein said motion-resistant particles and the force-driven particles are coated with a first sensing element and a second sensing element respectively, that can specifically bind to a target analyte in a test sample.

In a third aspect, the present disclosure relates to a system for detecting the presence or absence of an analyte in a test sample comprising:
a) a plurality of motion-resistant particles and a plurality of force-driven particles, wherein said motion-resistant particles are coated with a first sensing element and said force-driven particles are coated with a second sensing element, wherein both first and second sensing elements are capable of specific binding with the analyte, when present, to form bound motion-resistant/analyte/force-driven particle conjugates; and
b) a separation means comprising a viscous medium and a force that can separate said bound motion-resistant/analyte/force-driven particle conjugates, when present, from unbound motion-resistant particles and unbound force-driven particles.

In a fourth aspect, the present disclosure relates to use of the sensing kit or the system as described herein for virus and biomarker detection.

Advantageously, the use of the sensing kit or the system may be designed, for example, for virus detection for actual clinical trials using human sample. The use of force, which can be a mechanical force (such as magnetic force generated by the magnetic field), through the vicious medium for selection and sorting may minimize the possibility of false positive and false negative signals. The detection method is robust, fluorescence label-free, fast and can be used for home-based self-diagnostic.

### DEFINITIONS

The following words and terms used herein shall have the meaning indicated:

The term “conjugate” as used herein refers to an analyte particle that is linked to a motion-resistant particle and the analyte particle is also linked to a force-driven particle, to form a bound motion-resistant/analyte/force-driven particle.

The term “dimerization” or “dimerized” or “dimerize” as used herein refers to the formation of a linkage between two particles.

The term “non-specific binding” or ‘non-specific interaction” as used herein refers to dimerized particles not formed by the target analyte, which typically are caused by weak interactions that may be broken by application of a force.

The term “force-driven particle” refers to a particle that is able to be attracted (or repulsed) when a force is applied to the particle, leading to its movement in a medium (such as a viscous medium).

The term “motion-resistant particle” refers to a particle that does not move (in the unbound state) as a force is not applied on such particle. Where the motion-resistant particle forms a conjugate with an analyte and a force-driven particle, in view of the presence of the force-driven particle, such a conjugate can move in response to the force but at a slower speed due to the drag force applied from the viscous medium to the motion-resistant particle, whereby the movement speed of the conjugate is slower than that of unbound force-driven particles (since the motion-resistant particle resists or slows down the movement). The drag force applied to the motion-resistant particle also provides a tensile force to the analyte that binds to the force-driven particle on one site and the motion-resistant particle on another site.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a method of detecting the presence or absence of an analyte in a test sample will now be disclosed.

The method of detecting the presence or absence of an analyte in a test sample comprising the steps of:
a) incubating a plurality of motion-resistant particles and a plurality of force-driven particles with the test sample to form a test mixture, wherein the motion-resistant particles are coated with a first sensing element and the force-driven particles are coated with a second sensing element, both first and second sensing elements are capable of specific binding with the analyte, wherein when the analyte is present in the test sample, said analyte binds with said first and second sensing elements on said motion-resistant particles and said force-driven particles, respectively, to form bound motion-resistant/analyte/force-driven particle conjugates;
b) adding the test mixture of step a) to a viscous medium and applying a force to the force-driven particles to separate said bound motion-resistant/analyte/force-driven particle conjugates, when present, from the unbound motion-resistant particles and unbound force-driven particles.

In an embodiment, there is provided a method of detecting the presence or absence of an analyte in a test sample comprising the steps of:
a) incubating a plurality of non-magnetic particles and a plurality of magnetic particles with the test sample to form a test mixture, wherein the non-magnetic particles are coated with a first sensing element and the magnetic particles are coated with a second sensing element, both first and second sensing elements are capable of specific binding with the analyte, wherein when the analyte is present in the test sample, said analyte binds with said first and second sensing elements on said non-magnetic particles and said magnetic particles, respectively, to form bound non-magnetic/analyte/magnetic particle conjugates;
b) adding the test mixture to a viscous medium and applying a magnetic field to the magnetic particles to separate the bound non-magnetic/analyte/magnetic particle conjugates, when present, from the unbound non-magnetic particles and unbound magnetic particles.

In the above embodiment, the motion-resistant particle refers to the non-magnetic particle, the force-driven particle refers to the magnetic particle, the force refers to the magnetic force generated by an external magnetic field and the motion-resistant/analyte/force-driven particle conjugate refers to the non-magnetic/analyte/magnetic particle conjugate.

The test sample may be a liquid sample. The test sample may be derived or obtained from human, animal, microorganism, or environment. The test sample may comprise blood, urine, saliva, sputum, serum, liquid derived from cell, or tissue.

The analyte may be selected from the group comprising of virus, bacteria, archaea, fungus, protozoa, algae, deoxyribonucleic acid (DNA), ribonucleic acid (RNA), protein, antibody, antigen, cell, exosome, pathogen-associated molecule, pollutant, biomarker, target receptor, intracellular material, extracellular material, product derived from microorganism or modified biomaterial. The analyte may be multivalent. The analyte may be a virus. The analyte may be COVID-19 virus. The target receptor may be SARS-COV-2 receptor binding domain protein.

The method may further comprise step c) quantification of the bound motion-resistant/analyte/force-driven particle conjugates after separation of the bound motion-resistant/analyte/force-driven particle conjugates from the unbound motion-resistant particles and unbound force-driven particles. Quantification may be carried out by observing the bound motion-resistant/analyte/force-driven particle conjugates via a microscope or any suitable visualization device and counting the number of bound conjugates.

The size of the motion-resistant particles may be in the range of about 1 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 40 μm, about 1 μm to about 30 μm, about 1 μm to about 20 μm, about 1 μm to about 10 μm, about 1 μm to about 5 μm, about 5 μm to about 100 μm, about 10 μm to about 100 μm, about 20 μm to about 100 μm, about 30 μm to about 100 μm, about 40 μm to about 100 μm, about 50 μm to about 100 μm, about 60 μm to about 100 μm, about 70 μm to about 100 μm, about 80 μm to about 100 μm or about 90 μm to about 100 μm. The motion-resistant particle may be a non-magnetic particle.

The motion-resistant particles may be selected from the group consisting of polystyrene (PS), poly(lactic-co-glycolic acid) (PLGA), poly(methyl methacrylate) (PMMA), poly(c-caprolactone) (PCL), polyacrylate (PA), polylactide (PLA), poly(Glycidyl Methacrylate) (PGMA), melamine resin, silica, hydrogel and lipid membrane vesicle. The hydrogel may be spherical network of polyvinyl alcohol, acrylate polymers and copolymers with an abundance of hydrophilic groups, agarose, methylcellulose, hyaluronan, elastin like polypeptides. The motion-resistant particles may be any beads commonly used in analytical assays.

The size of the force-driven particles may be in the range of about 10 nm to about 50 μm, about 10 nm to about 10 μm, about 10 nm to about 1 μm, about 10 nm to about 500 nm, about 10 nm to about 100 nm, about 100 nm to about 500 nm, about 100 nm to about 1 μm, about 100 nm to about 10 μm. about 100 nm to about 50 μm, about 500 nm to about 1 μm, about 500 nm to about 10 μm, about 500 nm to about 50 μm, about 1 μm to about 10 μm, about 1 μm to about 50 μm, or about 10 μm to about 50 μm. The force-driven particle may be a magnetic particle.

When the force-driven particles are magnetic particles, the magnetic particles may be paramagnetic, superparamagnetic, or magnetic. The magnetic particles may be selected from the group consisting of iron, cobalt, nickel, alloy thereof, oxide thereof and combinations thereof.

The size of the motion-resistant particles may be preferably larger than the size of the force-driven particles. A larger sized motion-resistant particle may have a greater ability to resist motion.

The viscous medium may be transparent or translucent. The viscous medium may have a viscosity in the range of about 0.01 Pa·s to about 10 Pa·s, about 0.03 Pa·s to about 10 Pa·s, about 0.05 Pa·s to about 10 Pa·s, about 0.1 Pa·s to about 10 Pa·s, about 0.3 Pa·s to about 10 Pa·s, about 0.5 Pa·s to about 10 Pa·s, about 0.8 Pa·s to about 10 Pa·s, about 1 Pa·s to about 10 Pa·s, about 3 Pa·s to about 10 Pa·s, about 5 Pa·s to about 10 Pa·s, about 8 Pa·s to about 10 Pa·s, about 0.01 Pa·s to about 8 Pa·s, about 0.01 Pa·s to about 5 Pa·s, about 0.01 Pa·s to about 3 Pa·s, bout 0.01 Pa·s to about 1 Pa·s, about 0.01 Pa·s to about 0.8 Pa·s, about 0.01 Pa·s to about 0.5 Pa·s, about 0.01 Pa·s to about 0.3 Pa·s, about 0.01 Pa·s to about 0.1 Pa·s, about 0.01 Pa·s to about 0.05 Pa·s or about 0.01 Pa·s to about 0.03 Pa·s.

The viscous medium may be glycerol, olive oil, linseed oil, motor oil, syrup, ferrofluid, or their mixture thereof. The viscous medium may be serum or diluted serum. The viscous medium may be a mixture of glycerol and water. The weight percentage of glycerol in the mixture may be in the range of about 10 wt % to about 100 wt %, about 20 wt % to about 100 wt %, about 30 wt % to about 100 wt %, about 40 wt % to about 100 wt %, about 50 wt % to about 100 wt %, about 60wt % to about 100 wt %, about 70 wt % to about 100 wt %, about 80 wt % to about 100 wt %, about 90 wt % to about 100 wt %, about 10 wt % to about 90 wt %, about 10 wt % to about 80 wt %, about 10 wt % to about 70 wt %, about 10 wt % to about 60 wt %, about 10 wt % to about 50 wt %, about 10 wt % to about 40 wt %, about 10 wt % to about 30 wt % or about 10 wt % to about 20 wt %.

The viscous medium may be a solution of crowding agents. The crowding agents may be dextran, pegylated proteins, polyethylene glycol (PEG), lysozyme, bovine serum albumin (BSA), poly(sodium 4-styrene sulfonate) (PSS), hydrophilic polysccharide or their mixture thereof. The weight percentage of the crowding agents in the solution may be in the range of about 1 wt % to about 80 wt %, about 5 wt % to about 80 wt %, about 10 wt % to about 80 wt %, about 20 wt % to about 80 wt %, about 30 wt % to about 80 wt %, about 40 wt % to about 80 wt %, about 50 wt % to about 80 wt %, about 60 wt % to about 80 wt %, about 70 wt % to about 80 wt %, about 1 wt % to about 70 wt %, about 1 wt % to about 60 wt %, about 1 wt % to about 50 wt %, about 1 wt % to about 40 wt %, about 1 wt % to about 30 wt %, about 1 wt % to about 20 wt %, about 1 wt % to about 10 wt % or about 1 wt % to about 5 wt %. The solution may be an aqueous solution.

The viscous medium may be a base liquid with different types of thickening agents. The base liquid may be base oil. The thickening agent may be selected from polyurethanes, acrylic polymers, latex, styrene/butadiene, polyvinyl alcohol, natural clays, synthetic clays, cellulosics, sulfonates, gums, saccharides, proteins, organosilicones, polyethylene glycol, starch, alginic acid or alginate.

The first sensing element coated on the motion-resistant particles may be the same as the second sensing element coated on the force-driven particles. The analyte may have multiple equivalent binding sites for binding with the first sensing element and the second sensing element. The first sensing element coated on the motion-resistant particles may be different from the second sensing element coated on the force-driven particles. The analyte may preferably have different binding sites for the first sensing element and the second sensing element to reduce self-coupling of motion-resistant particles or self-coupling of force-driven particles and to enhance the sensitivity of detection. For a target analyte containing one binding site for one type of sensing element, and one binding site for the other type of sensing element, when the motion-resistant and the force-driven particles are coated with different sensing molecules targeting different binding sites on the target analyte, the dimerized particles can only be formed between motion-resistant and force-driven particles, resulting in the formation of specific conjugates.

The method of step a) may comprise steps of a1) incubating the motion-resistant or the force-driven particles with the test sample first to form a first incubation solution, and a2) further incubating said first incubation solution with the other force-driven or the other motion-resistant particles to form the bound motion-resistant/analyte/force-driven particle conjugates.

The incubation time for step a1) may be in the range of about 10 minutes to about 18 hours, about 10 minutes to about 12 hours, about 10 minutes to about 8 hours, about 10 minutes to about 4 hours, about 10 minutes to about 1 hour, about 10 minutes to about 40 minutes, about 10 minutes to about 30 minutes, about 10 minutes to about 20 minutes, about 20 minutes to about 18 hours, about 30 minutes to about 18 hours, about 40 minutes to about 18 hours, about 1 hour to about 18 hours, about 4 hours to about 18 hours, about 8 hours to about 18 hours or about 12 hours to about 18 hours.

The incubation time for step a2) may be about 10 minutes to about 2 hours, about 10 minutes to about 1.5 hours, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 2 hours, about 30 minutes to about 2 hours, about 40 minutes to about 2 hours, about 50 minutes to about 2 hours, about 1 hour to about 2 hours or about 1.5 hours to about 2 hours.

Advantageously, by separating the incubation steps a1) and a2), self-coupling between the motion-resistant particles or self-coupling between the force-driven particles can be minimized. The incubation time of step a1) is preferably longer than that of step a2) to enable thorough mixing of the motion-resistant or force-driven particles of step a1) with the analyte.

The method may further comprise, after step a1) and before step a2), a step of vortexing said first incubation solution to remove possible self-coupling of the motion-resistant particles or force-driven particles. The result of the method is the migration of motion-resistant particles along the direction of force applied to the force-driven particles, when they are linked to one or more force-driven particles due to the intervening analyte in the form of bound motion-resistant/analyte/force-driven particle conjugates.

After adding the test mixture to the viscous medium, the test mixture may form a transient interface between the test mixture and the viscous medium. Due to the high viscosity of the viscous medium, both the motion-resistant and force-driven particles will be kept near the interface.

When the force-driven particles used are magnetic particles, one or more magnets may be placed appropriately externally to or within the viscous medium to create a magnetic field to apply a magnetic force (as the mechanical force) onto the force-driven particle in a desired direction, which may drive separation of different particle species (unbound motion-resistant particles, unbound force-driven particles, and bound conjugates). Depending on the configuration of the detection system, the magnet(s) may be placed beneath the viscous medium to apply the magnetic force in a vertical direction or in the direction of the gravitational force. Under such a magnetic force, the unbound force-driven particles may move through the viscous medium the fastest, whereas the unbound motion-resistant particles may not move since it is not induced to movement by the magnetic force (which is much stronger than gravitational force). The specifically bound motion-resistant/analyte/force-driven particle conjugates may move along the direction of the magnetic force but may move significantly slower compared to the unbound force-driven particles due to greater inertia contributed by the bound motion-resistant particle in the conjugate and additionally due to the presence of the viscous medium. Therefore, these species may be separated in space, which may be easily detected.

The magnet(s) may be placed across the viscous medium for applying the magnetic force in a horizontal direction or in the direction perpendicular to the gravitational force. The unbound motion-resistant particles may remain in their original positions since they are not influenced by the magnetic force applied. The unbound force-driven particles may move towards the direction of the magnetic force applied. Similar to the case of magnetic force applied in a vertical direction, in the horizontal direction or in the direction perpendicular to the gravitational force the unbound force-driven beads may move the fastest, the unbound motion-resistant beads may not move, and the specifically bound motion-resistant/analyte/force-driven particle conjugates may move at a significantly slower speed compared to the unbound force-driven particles due to greater inertia contributed by the bound motion-resistant particle in the conjugate and additionally due to the presence of the viscous medium. Therefore, different species of particles may be separated and the specifically bound motion-resistant/analyte/force-driven particle conjugates may be observed by selecting the area of observation by a low-end optical microscope.

The force may be a mechanical force. The force may be an external force and may thus be an external mechanical force. The mechanical force may be magnetic force generated by the application of a magnetic field. Advantageously, mechanical force generated by means such as the magnetic field can be used as a selection tool to distinguish non-specific binding and specific binding, therefore reducing the possibilities of false positive signals. The specific interaction is the binding of the analyte to the motion-resistant particles and force-driven particles due to the presence of the first sensing element and the second sensing element to form the bound motion-resistant/analyte/force-driven particle conjugates. On the other hand, the non-specific interaction includes all other types of interactions such as hydrogen bonding, hydrophobic interaction, electrostatic interaction, etc. As non-specific binding is generally much weaker than specific interactions, particles bound based on non-specific binding will dissociate before the bound particles travel far. Only particle conjugates bound by specific interaction may achieve the separation, due to unlikeliness of dissociation under force.

When both specifically bound conjugates (i.e. motion-resistant/analyte/force-driven particle conjugates) and non-specifically bound conjugates (for example, motion-resistant/force-driven particle conjugates) move in the viscous medium due to application of the force on the force-driven particle in these conjugates, there may be concurrent drag force applied on the motion-resistant particle in these conjugates due to friction in the viscous medium. This may slow down the movement of both the specifically bound conjugates and the non-specifically bound conjugates compared to the unbound force-driven particles. In addition, this may be important for mechanically enhanced specificity, as the drag force applied on the motion-resistant particle in the specifically bound conjugates establishes a tensile force to the analyte such that in view of the specific binding of the analyte to the force-driven particle on one site and specific binding of the analyte to the motion-resistant particle on another site, the specifically bound conjugates are unlikely to dissociate as compared to non-specifically bound conjugates.

When the mechanical force is a magnetic force, the magnetic field generating the magnetic force may be applied for a period of about 10 seconds to about 60 minutes, about 10 seconds to about 30 minutes, about 10 seconds to about 10 minutes, about 10 seconds to about 1 minute, about 10 seconds to about 30 seconds, about 30 seconds to about 1 minute, about 30 seconds to about 10 minutes, about 30 seconds to about 30 minutes, about 30 seconds to about 60 minutes, about 1 minute to about 10 minutes, about 1 minute to about 30 minutes, about 1 minute to about 60 minutes, about 10 minutes to about 30 minutes, about 10 minutes to about 60 minutes, about 30 minutes to about 60 minutes, or as needed for the various particles to separate and where additionally, to quantify the amount of the bound motion-resistant/analyte/force-driven particle conjugates.

The depth of the viscous medium may be in the range of about 0.1 mm to about 10 mm, about 0.5 mm to about 10 mm, about 1 mm to about 10 mm, about 3 mm to about 10 mm, about 5 mm to about 10 mm, about 8 mm to about 10 mm, about 0.1 mm to about 8 mm, about 0.1 mm to about 5 mm, about 0.1 mm to about 3 mm, about 0.1 mm to about 1 mm or about 0.1 mm to about 0.5 mm, or can be adjusted to a wider depth as required for greater separation of the various unbound force-driven particles, unbound motion-resistant particles and bound motion-resistant/analyte/force-driven particle conjugates.

The observation results of the specifically bound motion-resistant/analyte/force-driven particle conjugates may be compared with a reference sample with non- specifically bound motion-resistant particle and force-driven particle pairs as control for quantification. The difference between the sample group and the control group may be used against a reference scale to determine the presence, amount of concentration of the analyte.

The method may further comprise a step of collecting the specifically bound motion-resistant/analyte/force-driven particle conjugates from the viscous medium after step b) or step c). The collection step may also be used as a concentrating step of the analyte. The collected samples may be used for further downstream processing such as sequencing.

The collection step may be achieved by flowing the viscous medium in a different direction from the magnetic field direction. The force (such as mechanical force) induced by the flow of the viscous medium may be used for further distinguishing the non-specific binding from the specific binding as two-dimensional sorting and selection. The flow of the viscous medium may be achieved by microfluidic channels, which facilitates detection and collection of specifically bound motion-resistant/analyte/force-driven particle conjugates.

Exemplary, non-limiting embodiments of a sensing kit, a system and the use of the sensing kit or the system will now be disclosed.

The sensing kit comprises
a plurality of motion-resistant particles; and
a plurality of force-driven particles.
wherein the motion-resistant particles and the force-driven particles are coated with a first sensing element and a second sensing element respectively, that can specifically bind to a target analyte in a test sample.

In one embodiment, there is provided a sensing kit comprising
a plurality of non-magnetic particles; and
a plurality of magnetic particles.
wherein the non-magnetic particles and the magnetic particles are coated with a first sensing element and a second sensing element respectively, that can specifically bind to a target analyte in a test sample.

In the above embodiment, the motion-resistant particle refers to the non-magnetic particle, and the force-driven particle refers to the magnetic particle.

The kit may further comprise a viscous medium. The viscous medium may be transparent or translucent. The viscous medium may have a viscosity about 0.1 Pa·s to about 10 Pa·s, about 0.3 Pa·s to about 10 Pa·s, about 0.5 Pa·s to about 10 Pa·s, about 0.8 Pa·s to about 10 Pa·s, about 1 Pa·s to about 10 Pa·s, about 3 Pa·s to about 10 Pa·s, about 5 Pa·s to about 10 Pa·s, about 8 Pa·s to about 10 Pa·s, about 0.1 Pa·s to about 8 Pa·s, about 0.1 Pa·s to about 5 Pa·s, about 0.1 Pa·s to about 3 Pa·s, bout 0.1 Pa·s to about 1 Pa·s, about 0.1 Pa·s to about 0.8 Pa·s, about 0.1 Pa·s to about 0.5 Pa·s or about 0.1 Pa·s to about 0.3 Pa·s.

The viscous medium may be glycerol, olive oil, linseed oil, motor oil, syrup, ferrofluid, or their mixture thereof. The viscous medium may be serum or diluted serum. The viscous medium may be a mixture of glycerol and water. The weight percentage of glycerol in the mixture may be in the range of about 10 wt % to about 100 wt %, about 20 wt % to about 100 wt %, about 30 wt % to about 100 wt %, about 40 wt % to about 100 wt %, about 50 wt % to about 100 wt %, about 60 wt % to about 100 wt %, about 70 wt % to about 100 wt %, about 80 wt % to about 100 wt %, about 90 wt % to about 100 wt %, about 10 wt % to about 90 wt %, about 10 wt % to about 80 wt %, about 10 wt % to about 70 wt %, about 10 wt % to about 60 wt %, about 10 wt % to about 50 wt %, about 10 wt % to about 40 wt %, about 10 wt % to about 30 wt % or about 10 wt % to about 20 wt %.

The size of the motion-resistant particles may be in the range of about 1 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 40 μm, about 1 μm to about 30 μm, about 1 μm to about 20 μm, about 1 μm to about 10 μm, about 1 μm to about 5 μm, about 5 μm to about 100 μm, about 10 μm to about 100 μm, about 20 μm to about 100 μm, about 30 μm to about 100 μm, about 40 μm to about 100 μm, about 50 μm to about 100 μm, about 60 μm to about 100 μm, about 70 μm to about 100 μm, about 80 μm to about 100 μm or about 90 μm to about 100 μm.

The size of the force-driven particles may be in the range of about 10 nm to about 50 μm, about 10 nm to about 10 about 10 nm to about 1 about 10 nm to about 500 nm, about 10 nm to about 100 nm, about 100 nm to about 500 nm, about 100 nm to about 1 μm, about 100 nm to about 10 μm, about 100 nm to about 50 μm, about 500 nm to about 1 μm, about 500 nm to about 10 μm, about 500 nm to about 50 μm, about 1 μm to about 10 μm, about 1 μm to about 50 μm, or about 10 μm to about 50 μm.

The kit may further comprise one or more magnets to apply a magnetic field that generates a magnetic force to separate the different particle species, when the force-driven particles are magnetic particles.

The system for detecting the presence or absence of an analyte in a test sample comprising:
a) a plurality of motion-resistant particles and a plurality of force-driven particles, wherein the motion-resistant particles are coated with a first sensing element and the force-driven particles are coated with a second sensing element, wherein both first and second sensing elements are capable of specific binding with the analyte, when present, to form bound motion-resistant/analyte/force-driven particle conjugates; and
b) a separation means comprising a viscous medium and a force that can separate said bound motion-resistant/analyte/force-driven particle conjugates, when present, from unbound motion-resistant particles and unbound force-driven particles.

In one embodiment, there is provided a system for detecting the presence or absence of an analyte in a test sample comprising:
a) a plurality of non-magnetic particles and a plurality of magnetic particles, wherein the non-magnetic particles are coated with a first sensing element and the magnetic particles are coated with a second sensing element, wherein both first and second sensing elements are capable of specific binding with the analyte, when present, to form bound non-magnetic/analyte/magnetic particle conjugates; and
b) a separation means comprising a viscous medium and a magnetic field that can separate said bound non-magnetic/analyte/magnetic particle conjugates, when present, from unbound non-magnetic particles and unbound magnetic particles.

In the above embodiment, the motion-resistant particle refers to the non-magnetic particle, the force-driven particle refers to the magnetic particle, the force refers to the magnetic field which generates a magnetic force and the motion-resistant/analyte/force-driven particle conjugate refers to the non-magnetic/analyte/magnetic particle conjugate.

The use of the sensing kit or the system as described herein for virus or biomarker detection.

Advantageously, the use of the sensing kit or the system may be specifically designed for virus or biomarker detection for actual clinical trials using human sample. The use of force (such as mechanical force, which in turn, can be a magnetic force generated by means such as a magnetic field) may minimize the possibility of false positive signals. The detection method is robust, fluorescence label-free, fast and can be used for home-based self-diagnostic.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1A is a schematic illustration depicting the general concept of the detection method of the present disclosure, where (1) denotes a sensing element, (2) denotes an analyte, (3) denotes a coated force-driven particle (in this case, a superparamagnetic bead coated with sensing element (1)), (4) denotes a coated motion-resistant particle (in this case, a non-magnetic bead coated with sensing element (1)), (5) denotes bound motion-resistant/analyte/force-driven particle conjugate, (6) denotes a highly viscous medium and (7) denotes a pair of magnets.

FIG. 1B is a schematic illustration depicting two-dimensional detection method of the present disclosure for analyte detection and collection, which is based on the general concept of FIG. 1A coupled with the flow of viscous medium at a velocity v. The labelling of the components are as follows: (1) denotes a sensing element, (2) denotes an analyte, (3) denotes a coated force-driven particle (in this case, a magnetic bead coated with sensing element (1)), (4) denotes a coated motion-resistant particle (in this case, a non-magnetic bead coated with sensing element (1)), (5) denotes bound motion-resistant/analyte/force-driven particle conjugate and (6) denotes a highly viscous medium.

FIG. 2 is a schematic illustration of a proof-of-concept experimental procedure using double-stranded DNA with biotin and digoxigenin labelling as analyte. The labelling of the components are as follows: (100) denotes a double-stranded DNA with biotin and digoxigenin labelling as analyte, (200) denotes a 10 μm polystyrene bead coated with streptavidin, (300) denotes a 3 μm superparamagnetic bead coated with streptavidin, (400) denotes a buffer solution, (500) denotes a glycerol viscous medium, (600) denotes an interface between the buffer solution and the glycerol viscous medium, (700) denotes the motion-resistant/analyte/force-driven particle conjugate, (800) denotes a pair of magnets, and (900) denotes a microscope.

FIG. 3 is a schematic illustration depicting the detection of digoxigenin-coated 200 nm mock virus as analyte (1000) with motion-resistant, large anti-digoxigenin coated polystyrene bead (2000) and small force-driven, anti-digoxigenin coated superparamagnetic bead (3000). The analyte (1000) thus dimerizes with the coated large anti-digoxigenin polystyrene bead (i.e. coated non-magnetic bead) (2000) and the coated small anti-digoxigenin coated superparamagnetic bead (3000) to form bound motion-resistant/analyte/force-driven particle conjugate (4000).

FIG. 4 is the video imaging results of the proof-of-concept experiment of FIG. 2 where double-stranded DNA with biotin and digoxigenin labelling is the analyte to be detected.

FIG. 5 is the experimental results comparing the number of polystyrene beads detected using different concentrations of analyte (0.4 pM and 4 fM) for the proof-of-concept experiment of FIG. 3 where double-stranded DNA with biotin and digoxigenin labelling is the analyte to be detected. The number of polystyrene beads counted in experiment Trial 1 and 2 showed 121 times increase and 0.9 times increase for the 0.4 pM and 4 fM analyte concentrations as compared to without analyte respectively.

FIG. 6 is the experimental results comparing the number of polystyrene beads detected using different concentrations of analyte (6 pM, 60 fM and 600 fM) in standard buffer for the experiment of FIG. 3, with additional results for using the 600 fM analyte in 10 times diluted human serum. The number of polystyrene beads counted showed 3 times increase, 32 times increase, and 156 times increase for 60 fM, 600 fM and 6 pM analyte concentrations in standard buffer as compared to without analyte respectively. When 10 times human serum is used instead of standard buffer, under 600 fM analyte concentration, the number of polystyrene beads counted showed 39 times increase as compared to without analyte.

### DETAILED DESCRIPTION OF DRAWINGS

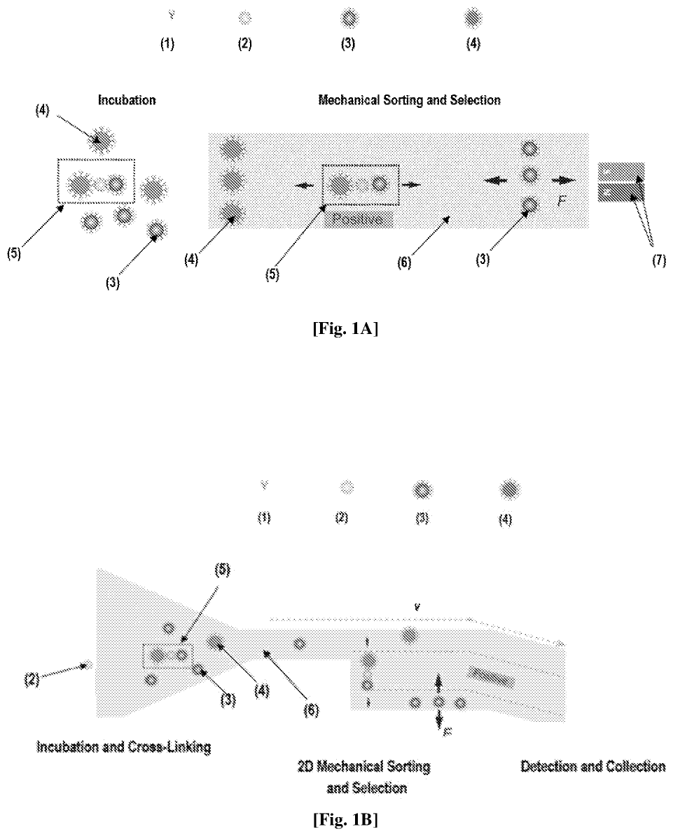

FIG. 1A is a schematic illustration depicting the general concept of the detection method of the present disclosure. Without the analyte (2), the beads would not dimerize to form the bound motion-resistant/analyte/force-driven particle conjugate (5). After a certain period of incubation, the mixture was subjected to highly viscous medium (6) with magnets (7) placed aside. All force-driven particles (superparamagnetic beads) (3) in the mixture were then attracted and travel towards the direction where the force is induced. When the superparamagnetic bead dimerized with the motion-resistant particle (non-magnetic bead in this case) in the presence of the analyte (5), due to larger size of dimerized beads, the dimerized beads experienced larger drag force in the viscous medium (6) and travelled slower than the unbound superparamagnetic beads (3). Unbound non-magnetic beads (4) on the other hand stayed still in the medium. That created separation of different species of beads for easy quantification of the analyte (2). The drag force between the dimerized beads also provided a gating mechanism to screen off the non-specific bindings. As non-specific binding is generally much weaker than specific interactions, beads dimerized based on non-specific binding would dissociate before the dimer travels far. Only specific interaction-based bead dimer (5) could achieve the separation.

FIG. 1B is a schematic illustration depicting two-dimensional detection method of the present disclosure for analyte detection and collection, which is based on the general concept of FIG. 1A coupled with the flow of viscous medium at a velocity v, such that the analyte (2) was pre-incubated with the sensing elements (1) coated motion-resistant (non-magnetic) bead (4) and force-driven (magnetic) bead (3) to allow forming specifically bound motion-resistant/analyte/force-driven particle conjugates (5), which were then loaded into a sorting and collecting channel containing a viscous medium (6) where a force F (in this case a magnetic force) was applied in the vertical direction to separate the specifically bound motion-resistant/analyte/force-driven particle conjugates (5) from the individual non-bound beads. An additional flow of the viscous medium is introduced in a different direction from the direction of the induced force (i.e., magnetic field direction) and at a velocity of v to facilitate analyte detection and collection (in this case along the direction labelled as “positive direction” towards to the end of the separator).

### EXAMPLES

Non-limiting examples of the invention will be further described in greater details by reference to specific Examples, which should not be construed as in any way limiting the scope of the invention.

### Example 1: Detecting Dual-Labelled Double-Stranded DNA

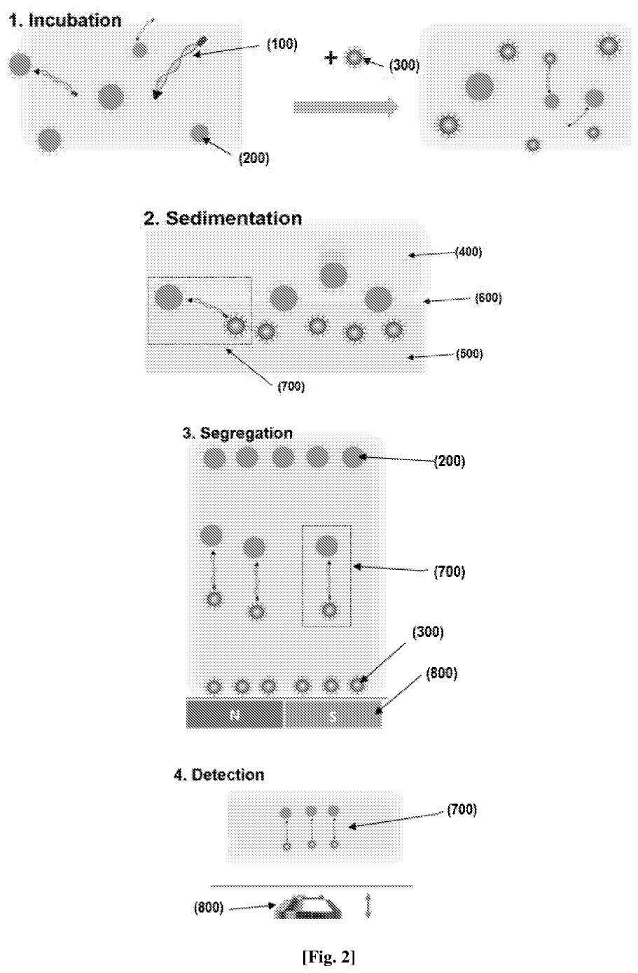

To demonstrate the feasibility of detection, a proof-of concept experiment was performed as shown in FIG. 2 where double-stranded DNA with biotin and digoxigenin labelled at two ends (ThermoFisher, US) as the analyte (100) was used to induce bead dimerization. The analyte (100) was designed to dimerize the anti-digoxigenin antibody (Pierce, US)-coated 10 μm polystyrene beads (Polysciences, US) (200) and the streptavidin (Pierce, US)-coated 3 μm superparamagnetic beads (Invitrogen, US) (300). Here, the motion-resistant particle is the polystyrene bead (which is non-magnetic) and the force-driven particle is the superparamagnetic bead (which is magnetic).

Firstly, as shown in the “1. Incubation” step of FIG. 2, the analyte (100) at 0.4 picomolar (pM) and 4 femtomolar (fM) concentrations were incubated with motion-resistant polystyrene beads (200) for 30 minutes and 10 hours respectively to allow binding of the analyte (100) to the polystyrene beads (200). Then excessive streptavidin-coated superparamagnetic beads (300) were added into the buffer and incubated a further 30 minutes to maximize the chance of bead dimerization.

Secondly, as shown in the “2. Sedimentation” step of FIG. 2, the resultant mixture from step 1 containing the bound motion-resistant/analyte/force-driven particle conjugate (700) was added atop a viscous medium (500), such as glycerol (Fisher Chemical, US), in the channels, forming a transient interface (600) between the buffer solution (400) of the resultant mixture and glycerol (500). Here, the bound motion-resistant/analyte/force-driven particle conjugate is bound polystyrene/analyte/superparamagnetic bead conjugate. Due to the high viscosity of the medium and small gravitational force, both bead species are kept near the interface.

Following that, a pair of magnets (Supermagetman, US) (800) is placed beneath the glycerol channels for approximately 10 minutes. Magnetic force drove separation of bead species as shown in the “3. Segregation” step of FIG. 2.

The channels were then put under a microscope (Olympus IX41) (900) using 10× and 50× objective lens as shown in the “4. Detection” step of FIG. 2. By adjusting the focus height of objective, large polystyrene beads at bottom region of the glycerol, which are in fact the bound motion-resistant/analyte/force-driven particle conjugate (700), can be observed and counted.

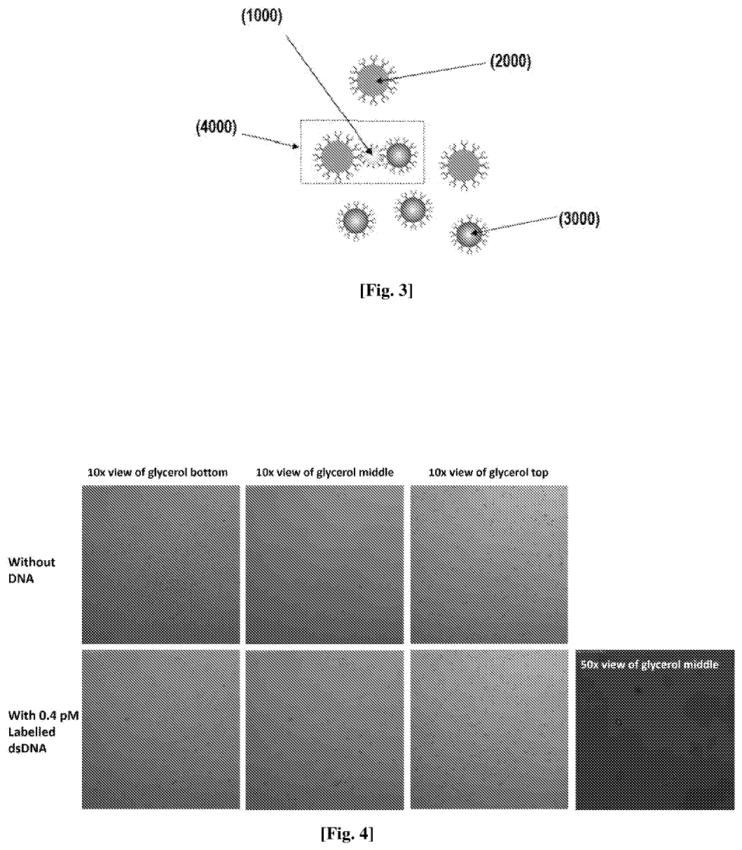

Following the previous procedures, videos of scanning through the glycerol were generated. Several frames of the video are listed in FIG. 4, to demonstrate the successful separation of bead species and therefore the detection of analyte in the buffer. In the control experiment without any analyte added, under 10× magnification, 3 μm superparamagnetic beads were seen scattering over the bottom region of glycerol, with only one 10 μm polystyrene bead visible. Moving up the focus, neither superparamagnetic beads nor polystyrene beads can be observed. Further focusing up to the glycerol top region, a layer of polystyrene beads can be observed. Compared to the control experiment without analyte, in the presence of only 0.4 pM analyte, over a hundred polystyrene beads can be observed in the glycerol bottom and middle regions. Under 50× magnification, the large polystyrene beads can be always seen companied by one or more small magnetic beads, in a dimerization or oligomerization form.

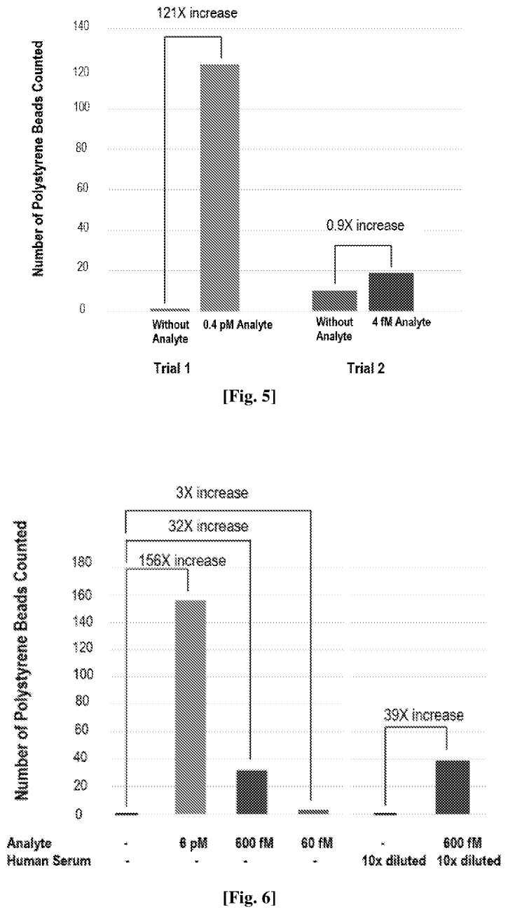

The detailed quantifications of polystyrene bead number in the glycerol bottom and middle regions are shown in FIG. 5 for both experimental trials in the presence of 0.4 pM analyte (with 30 min incubation time) and 4 fM analyte (with overnight incubation). Stark contrast can be seen from their respective control experiments, where the analyte was absent. This experiment serves as the initial proof-of-concept for the method in the present disclosure and demonstrates the limit of detection in said method can be as low as femtomolar concentration level.

### Example 2: Detecting Digoxigenin-Coated Mock Virus

The capability of the present method in virus detection was also tested. Procedures followed were similar to what has been described in Example 1, except that the type of analyte was replaced with digoxigenin-coated 200 nm latex beads (Bangs Lab, US) as mock virus particles (1000), and accordingly, the large polystyrene beads (2000) and small superparamagnetic beads (3000) were all anti-digoxigenin coated, as shown in FIG. 3. The analyte (1000) was thus capable of dimerizing the anti-digoxigenin coated large polystyrene beads (2000) and the anti-digoxigenin coated small superparamagnetic beads (3000), to form bound motion-resistant/analyte/force-driven particle conjugate (4000). Here, the motion-resistant particle is the polystyrene bead (which is non-magnetic), the force-driven particle is the superparamagnetic bead (which is magnetic) and the bound motion-resistant/analyte/force-driven particle conjugate is bound polystyrene/analyte/superparamagnetic bead conjugate.

Similar to the results for the dual-labelled DNA analyte detection, when 6 pM, 600 fM and 60 fM mock virus (analyte, (1000)) in PBS standard buffer were included in the sample for detection, sharp increase of signal can be observed, giving 156 times, 32 times, and 3 times increase respectively compared to the signal from the control where the mock virus analyte was absent, as shown in FIG. 6. The result further strengthens the reliability of the detection method. In addition, to mimic the actual diagnosis, the detection was also performed in 10 times diluted human serum. It is clear that the signal generated in such condition does not deviate from the sample with the same concentration of mock virus, that is 600 fM concentration, in PBS standard buffer, demonstrating the robustness of detection method in the complex environment like human serum. Based on this result, the method disclosed in the present invention may be easily applied in the clinical setting, to generate accurate diagnosis of virus in human biological samples.

### INDUSTRIAL APPLICABILITY

The method, sensing kit and system as disclosed herein may be used in a wide variety of diagnostic applications such as environmental monitoring, clinical testing and home-based self-diagnostic, for the of detection of the presence or absence of an analyte in a test sample. The method, sensing kit and system offer single-particle sensitivity and enhanced specificity based on mechanically gated selection of specific interactions. Further, the method allows collection or concentration of the targeted analyte for downstream processing.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of detecting a presence or absence of an analyte in a test sample comprising the steps of:
a) incubating a plurality of motion-resistant particles and a plurality of force-driven particles with said test sample to form a test mixture, wherein said motion-resistant particles are coated with a first sensing element and said force-driven particles are coated with a second sensing element, both first and second sensing elements are capable of specific binding with the analyte, wherein when the analyte is present in the test sample, said analyte binds with said first and second sensing elements on said motion-resistant particles and said force-driven particles, respectively, to form bound motion-resistant/analyte/force-driven particle conjugates; and
b) adding the test mixture of step a) to a viscous medium and applying a force to the force-driven particles to separate said bound motion-resistant/analyte/force-driven particle conjugates, when present, from the unbound motion-resistant particles and unbound force-driven particles.

2. The method of claim 1 further comprising step c) quantification of the bound motion-resistant/analyte/force-driven particle conjugates after separation of the bound motion-resistant/analyte/force-driven particle conjugates from the unbound motion-resistant particles and unbound force-driven particles.

3. The method of claim 1, wherein a size of said motion-resistant particles is in a range of 1 μm to 100 μm.

4. The method of claim 1, wherein said motion-resistant particles are selected from the group consisting of polystyrene (PS), poly(lactic-co-glycolic acid) (PLGA), poly(methyl methacrylate) (PMMA), poly(c-caprolactone) (PCL), polyacrylate (PA), polylactide (PLA), poly(Glycidyl Methacrylate) (PGMA), melamine resin, silica, hydrogel and lipid membrane vesicle.

5. The method of claim 1, wherein a size of said force-driven particles is in a range of 10 nm to 50 μm.

6. The method of claim 1, wherein when said force-driven particles are magnetic particles said magnetic particles are selected from the group consisting of iron, cobalt, nickel, alloy thereof, oxide thereof and combinations thereof.

7. The method of claim 1, wherein said viscous medium has a viscosity in a range of 0.01 Pa·s to 10 Pa·s; and wherein said viscous medium is selected from the group consisting of glycerol, olive oil, linseed oil, motor oil, syrup, ferrofluid, a solution of crowding agents, a base liquid with one or more thickening agents, and mixture thereof.

8. The method of claim 1, wherein said analyte has multiple equivalent binding sites for binding with said first sensing element and said second sensing element; and wherein said binding sites are different for the first sensing element and second sensing element.

9. The method of claim 1, wherein step a) comprise the steps of
a1) incubating said motion-resistant or said force-driven particles with said test sample first to form a first incubation solution, and
a2) further incubating said first incubation solution with the other force-driven particles or other motion-resistant particles to form the bound motion-resistant/analyte/force-driven particle conjugates.

10. The method of claim 9, further comprising, after step a1) and before step a2), a step of vortexing said first incubation solution.

11. The method of claim 9, wherein an incubation time for step a1) is in a range of 10 minutes to 18 hours; and wherein an incubation time for step a2) is in a range of 10 minutes to 2 hours.

12. The method of claim 11, wherein the incubation time for step a1) is longer than the incubation time for step a2).

13. The method of claim 1, wherein said force is applied vertically or horizontally or both vertically and horizontally across said viscous medium.

14. The method of claim 1, wherein said force is applied for a duration of 10 seconds to 60 minutes.

15. The method of claim 1, further comprising a step of collecting said bound motion-resistant/analyte/force-driven particle conjugates from said viscous medium after step b).

16. A sensing kit comprising:
a plurality of motion-resistant particles; and
a plurality of force-driven particles,
wherein said motion-resistant particles and the force-driven particles are coated with a first sensing element and a second sensing element respectively, that can specifically bind to a target analyte in a test sample.

17. A system for detecting a presence or absence of an analyte in a test sample comprising:
a) a plurality of motion-resistant particles and a plurality of force-driven particles, wherein said motion-resistant particles are coated with a first sensing element and said force-driven particles are coated with a second sensing element, wherein both first and second sensing elements are capable of specific binding with the analyte, when present, to form bound motion-resistant/analyte/force-driven particle conjugates; and
b) a separation means comprising a viscous medium and a force that can separate said bound motion-resistant/analyte/force-driven particle conjugates, when present, from unbound motion-resistant particles and unbound force-driven particles.

18. (canceled)
